(19) United States
(12) Patent Application Publication
(10) Pub. No.: US 2025/0032377 A1
(43) Pub. Date: Jan. 30, 2025

(54) Oral Care Compositions Comprising Dicarboxylic Acid

(71) Applicant: The Procter & Gamble Company, Cincinnati, OH (US)

(72) Inventors: Andrew Frederic Groth, Mason, OH (US); Paul Albert Sagel, Maineville, OH (US); Samuel James St. John, Cincinnati, OH (US)

(21) Appl. No.: 18/911,823

(22) Filed: Oct. 10, 2024

Related U.S. Application Data
(63) Continuation-in-part of application No. 17/308,080, filed on May 5, 2021.
(60) Provisional application No. 63/020,035, filed on May 5, 2020.

(51) Int. Cl. A61K 8/362; A61K 8/21; A61K 8/25; A61Q 11/00

(52) U.S. Cl. CPC A61K 8/362; A61K 8/21; A61K 8/25; A61Q 11/00; A61K 2800/28; A61K 2800/30

(57) ABSTRACT

An oral care composition includes a dicarboxylic acid or a salt thereof, a fluoride ion source, and silica abrasive. The composition may have a pH in a range of from about 4 to about 5.5. The oral care composition, after 12 months of aging at about 20° C., may have a soluble fluoride level that is more than 50% of a theoretical amount of fluoride.

### FIELD OF THE TECHNOLOGY

The present disclosure relates generally to oral care compositions comprising dicarboxylic acid and fluoride and, more particularly, to oral care compositions with unexpectedly improved fluoride stability.

### BACKGROUND

Oral care compositions, such as toothpaste and/or dentifrice compositions, can be applied to the oral cavity to clean and/or maintain the aesthetics and/or health of the teeth, gums, and/or tongue. Many oral care compositions are used to deliver active ingredients, e.g., Sn and F, directly to oral care surfaces. For example, oral care compositions have included antimicrobial agents, such as tin ions, to counter oral bacteria and to prevent and treat conditions caused by bacteria in the oral cavity, such as formation of dental plaque and calculus. The formation of dental plaque and calculus and failure to stop their proliferation are the primary cause of dental caries, gingivitis, periodontal disease, and tooth loss. Additionally, tin ions can deposit on surfaces in the oral cavity to provide protective functions, such as antierosion or antisensitivity benefits.

However, tin can be challenging to properly formulate in oral care compositions due to reactivity between tin and other components of oral care compositions. Under-stabilizing or over-stabilizing tin can lead to lower availability of tin ions to provide the desired benefit. For example, if the tin is under-stabilized, the tin can react with other components of the oral care composition, such as silica, water, etc., which can lead to a lower amount of available tin ions. In contrast, if the tin is over-stabilized or the chelant-tin chelation is too strong, the tin ions will be tied up while in the oral cavity, which can also lead to a lower amount of bioavailable tin ions to produce the desired oral care benefit. Delivery of Sn and fluoride to the oral hard and soft tissue surfaces is, therefore, important.

Toothpaste compositions can also include a fluoride source that helps the teeth resist plaque acids for the prevention of cavities. Additionally, many oral care compositions can be used to remove and/or prevent stains on oral cavity surfaces. Whitening of oral care hard tissue surfaces can occur through chemical or physical means. Physical agents include the combination of a brush and abrasive. Chemical agents include oxidizing agents (e.g., peroxide), anticalculus agents (e.g., polyphosphates), or other agents capable of dislodging surface stains through chemical action (e.g., bicarbonates).

Each chemical whitening agent has its drawbacks. Oxidizing agents are challenging to keep from reacting with other ingredients of the oral care composition during the composition's lifecycle. Additionally, they are not reactive with some surface stains; thereby, not fulfilling their primary purpose. Polyphosphate-based anticalculus agents are highly susceptible to hydrolysis breaking down in compositions to ineffective orthophosphate. In the presence of soluble fluoride, the breakdown can be accelerated resulting in non-bioavailable fluoride. Oxidizing agents can also be irritating to the oral soft tissues. Some other chemical agents have characteristic tastes that make them unpleasant to consumers. Bicarbonate-based toothpastes tend to taste like baking soda whose unique experience is not enjoyed by a large segment of consumers. In total, existing whitening agents can be challenging to formulate with for a variety of reasons specific to each agent.

Thus, there is a need for an oral care composition comprising a whitening agent, which can effectively remove and prevent the accumulation of stain, while improving existing formulation challenges.

### SUMMARY

In an embodiment, an oral care composition is provided and includes from about 0.1% to about 15%, by weight of the composition, of a dicarboxylic acid or a salt thereof, a fluoride ion source, and silica abrasive. The composition has a pH in a range of from about 4 to about 5.5. The oral care composition, after 12 months of aging at about 20° C., may have a soluble fluoride level that is more than 50% of a theoretical amount of fluoride.

In another embodiment, an oral care composition is provided and includes from about 0.1% to about 15%, by weight of the oral care composition, of oxalic acid or a salt thereof, a fluoride ion source that includes sodium monofluorophosphate, stannous fluoride, or a combination thereof, and silica abrasive. The oral care composition, after 12 months of aging at about 20° C., may have a soluble fluoride level that is more than 60% of the theoretical amount of fluoride.

In another embodiment, an oral care composition is provided and includes from about 0.1% to about 15%, by weight of the composition, of dicarboxylic acid or a salt thereof, from about 0.01% to about 10%, by weight of the composition, of a tin ion source, sodium fluoride, and silica abrasive. The composition has a pH in a range of from about 4 to about 5.5.

In another embodiment, an oral care composition is provided and includes from about 0.1% to about 15%, by weight of the composition, of malonic acid or a salt thereof, sodium fluoride, and silica abrasive. The composition has a pH in a range of from about 4 to about 5.5.

### DETAILED DESCRIPTION

Embodiments of the present invention are directed to oral care whitening compositions that have dicarboxylic acid, such as oxalic acid, malonic acid, methylmalonic acid, tartronic acid, maleic acid, or combinations thereof, and unexpectedly improve fluoride stability in a particular acidic pH range. Dental stain, or tooth stain, is caused by the cation-crosslinked proteins and extracellular polysaccharides that then act as reservoirs for colored porphyrins and organic and/or inorganic chromophores. Cross-linking can occur electrostatically via charge-charge, dipole-dipole, and/or dipole-charge interactions. Interrupting these electrostatic forces can facilitate stain removal. The resulting compositions according to embodiments of the present invention provide efficacious oral hard tissue whitening benefits with fewer drawbacks that are observed with other whitening agents.

At low pH and in the presence of silica abrasives (i.e., the conditions necessary to improve stain removal in combination with a dicarboxylate anion), fluoride can be reactive with silica to form non-bioavailable fluorosilicates. Additionally, at low pH and in the presence of silica abrasives with transition metal impurities, fluoride can be reactive with transition metals to form insoluble transition-metal-fluoride salts yielding non-bioavailable fluoride. Without wishing to be bound by theory, it is believed that the disclosed oral care compositions according to the embodiments of the present invention provide unexpectedly high fluoride stability.

Further embodiments of the present invention are directed to oral care compositions that have an optimum stabilizing system for delivering a high amount of bioavailable Sn to the enamel surface while providing for soluble tin stability. The resulting invention provides efficacious oral hard tissue erosion prevention to less optimally stabilized systems.

Although the initial process related to both caries and dental erosion begins with teeth being subjected to acid attack, the subsequent stages of each process are quite distinct. Dental erosion is a process that generally initiates on facial surfaces of teeth on which plaque is not present, while caries occurs under plaque-coated surfaces, where relatively constant, low-level acid challenges penetrate through the surface of the teeth and create subsurface lesions while allowing the surface to remain intact. In the case of dental erosion, excessive exposure to dietary acids causes the surfaces of the teeth then begin to soften resulting in tooth loss. Although fluoride is recognized to strengthen acid damaged enamel during remineralization, fluoride provides poor protection against dietary acids.

SnF₂ is a well-established anticaries agent that is unique among the fluoride sources used in over-the-counter dentifrices because of the stannous ion. Stannous deposits onto the tooth surface at both calcium and phosphate sites forming a thin layer of insoluble mineral precipitates. These precipitates slow the attack of dietary acids thus helping to prevent the loss of enamel to erosive processes. The ability of stannous to reach and then react with the enamel surface depends upon the choice of stabilizers in the oral care composition.

The unique properties of small molecule mono-, di-, tri-, and tetra-carboxylic acids, like gluconic acid, oxalic acid, and citric acid, allow them to be highly effective stabilizing ligands in a particular pH range. A specific combination of these small-molecule stabilizers allows for adequate shelf stability and high bioavailability. While not wishing to be bound by theory, it is believed that the disclosed oral care compositions of the present invention provide an unexpectedly high crosion benefit in comparison to less well stabilized compositions defined by an optimum ratio of Sn to mono- to poly-carboxylic acid stabilizers.

Additionally, while the use of cationic antimicrobial agents can provide many benefits when applied to the oral cavity, as described herein, cationic antimicrobial agents can also contribute to surface staining on teeth. Oral hard surface stains can be caused by interaction between (1) cation-crosslinked proteins and/or extracellular polysaccharides and (2) colored porphyrins and organic and/or inorganic chromophores, such as metal ions, which can form a colored matrix. Disrupting this colored matrix can facilitate stain removal.

Chemical whitening agents loosen the interactions of the compounds in this colored matrix to dislodge it from the oral hard tissue surfaces. While not wishing to be bound by theory, it is believed that chemical whitening agents, such as dicarboxylic acid, can act as solubilizing ligands for the porphyrins and/or chromophores to remove stain from the surface. Furthermore, manipulating the pH and ionic strength of the disclosed oral care composition can further reduce the strength of the electrostatic bonds by protonating anionically charged moieties or by reducing the potential of the electrostatic double layer further facilitating the solubilization of cationic moieties by solubilizing ligands.

In total, the unique properties of dicarboxylic acid compounds, such as oxalic acid, allow them to be highly effective stabilizing ligands for Sn in a particular pH range. As such, oral care compositions as disclosed herein can provide an unexpectedly high whitening benefit in a pH range in which convention whitening agents cannot be used whilst also providing unexpectedly high stability and delivering Sn and F to oral hard tissues.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen’ or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise dicarboxylic acid, tin, and/or fluoride. Additionally, the oral care compositions can comprise other optional ingredients, as described below. The section headers below are provided for convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

### Dicarboxylic Acid

The oral care composition comprises dicarboxylic acid. The dicarboxylic acid comprises a compound with two carboxylic acid functional groups. The dicarboxylic acid can comprise a compound or salt thereof defined by Formula I.

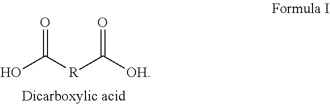

R can be null, alkyl, alkenyl, allyl, phenyl, benzyl, aliphatic, aromatic, polyethylene glycol, polymer, O, N, P, or combinations thereof.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, fumaric acid, terephthalic acid, glutaric acid, salts thereof, or combinations thereof. The dicarboxylic acid can comprise suitable salts of dicarboxylic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

The oral care composition can comprise from about 0.0001% to about 25%, from about 0.1% to about 15%, from about 0.01% to about 10%, or from about 1% to about 5%, of dicarboxylic acid.

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

After 12 months of aging at about 20° C., the oral care composition may have a soluble fluoride level that is more than 50%, more than 60%, more than 70%, more than 75%, more than 80%, or more than 90% of a theoretical amount of fluoride. After 6 months of aging at about 40° C., the oral care composition may have a soluble fluoride level that is more than 50%, more than 60%, more than 70%, more than 75%, more than 80%, or more than 90% of a theoretical amount of fluoride.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

The oral care composition of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, or from about 0.3% to about 0.6%, by weight of the dentifrice composition. Alternatively, the oral care composition can be essentially free of, substantially free of, or free of zinc.

### pH

The pH of the oral care compositions as described herein can be from about 4 to about 7, from about 4 to about 6, from about 4.5 to about 6.5, or from about 4.5 to about 5.5. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

The pH of the oral care compositions as described herein have a preferred pH of below about 7 or below about 6 due to the pKa of the dicarboxylic acid. While not wishing to be bound by theory, it is believed that the dicarboxylic acid displays unique behavior when the pH is below about 7 or below about 6, but surfaces in the oral cavity can only also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential to damage teeth by acid dissolution is greatly increased. Consequently, the oral care compositions comprising dicarboxylic acid, as described herein, preferably have a pH from about 4 to about 7, from about 4 to about 6, from about 4.5 to about 6.5, or from about 4.5 to about 5.5 to minimize metal hydroxide/metal oxide formation and any damage to oral hard tissues (enamel, dentin, and cementum).

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

### Monodentate Ligand

The oral care composition can comprise monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include from about 0.01% to about 10%, from about 0.1% to about 15%, from about 1% to about 5%, or from about 0.0001 to about 25%, by weight of the composition, of the monodentate ligand.

### Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e. with two functional groups), tridentate (i.e. with three functional groups), tetradentate (i.e. with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof. The polydentate ligand can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, tartaric acid, citric acid, phytic acid, pyrophosphate, tripolyphosphate, tetrapolyphosphate, hexametaphoshate, salts thereof, and/or combinations thereof. The oral care composition can include from about 0.01% to about 10%, from about 0.1% to about 15%, from about 1% to about 5%, or from about 0.0001 to about 25%, by weight of the composition, of the polydentate ligand.

### Ratio of Tin to Monodentate Ligand to Polydentate Ligand

The oral care composition, as described herein, can comprise a ratio of tin to monodentate ligand to polydentate ligand that provides an unexpectedly high amount of soluble tin and/or a superior fluoride uptake. Suitable ratios of tin to monodentate ligand to polydentate ligand can be from about 1:0.5:0.5 to about 1:5:5, from about 1:0.5:0.75 to about 1:5:5, from about 1:1:1 to about 1:5:5, from about 1:1:0.5 to about 1:2.5:2.5, from about 1:1:1 to about 1:2:2, from about 1:0.5:0.5 to about 1:3:1, or from about 1:0.5:0.5 to about 1:1:3.

Desired herein are oral care compositions with a soluble Sn of at least about 1000 ppm, 2000 ppm, 4000 ppm, at least about 4500 ppm, at least about 5000 ppm, at least about 6000 ppm, and/or at least about 8000 ppm. Also desired herein are oral care compositions with a fluoride uptake of at least about 6.5 μg/cm², at least about 7.0 μg/cm², at least about 8.0 μg/cm², or at least about 9.0 μg/cm² after a time period of at least about 9 days, 30 days, 65 days, 75 days, 100 days, 200 days, 365 days and/or 400 days.

In total, while not wishing to be bound by theory it is believed that the soluble Sn amount is correlated to bioavailable Sn as it is freely available to provide an oral health benefit. Fully bound Sn (i.e. Sn that is overchelated) or precipitated Sn (i.e. insoluble tin salts, such as Sn(OH)₂ and/or Sn-based stains can form when Sn is underchelated) would not be included in the measurement for soluble Sn. Additionally, while not wishing to be bound by theory, it is believed that a carefully balanced ratio of Sn to monodentate and polydentate ligands can provide a high amount of bioavailable fluoride and Sn ions without some of the negatives to the use of cationic antimicrobial agents, such as surface staining. Thus, additional screening experiments were done to quantify and qualify the ranges and identities of monodentate and polydentate ligands.

### Thickening Agent

The oral care composition can comprise one or more thickening agents. Thickening agents can be useful in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Abrasive

The oral care composition of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

The oral care composition comprising silica can comprise silica with residual metals. Those residual metals can comprise aluminum, calcium, or iron. The residual aluminum can comprise up to about 100 ppm, up to about 500 ppm, up to about 1000 ppm, or up to about 5000 ppm of total aluminum in the silica. The residual calcium can comprise up to about 10 ppm, up to about 50 ppm, up to about 100 ppm, or up to about 500 ppm of total calcium in the silica. The residual iron can comprise up to about 100 ppm, up to about 250 ppm, or up to about 500 ppm of the total iron in the silica.

### Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula II, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

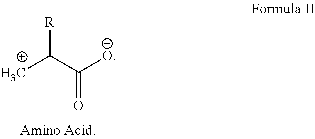

R is any suitable functional group

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acid which has an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e., uncomplexed). If the neutral amino acid is in its salt form, suitable salts include salts known in the art to be pharmaceutically acceptable salts considered to be physiologically acceptable in the amounts and concentrations provided.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, Pthalamidoperoxycaproic acid, or mixtures thereof.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), crythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions for the delivery of the dicarboxylic acid include emulsion compositions, such as the emulsions compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Oral Care Regimen

The dicarboxylic acid can be delivered in the same composition as the tin and/or fluoride or the dicarboxylic acid can be delivered in a separate composition. For example, a first composition can comprise tin and/or fluoride and a second composition can comprise dicarboxylic acid. The first and second composition can be delivered simultaneously, such as in a dual-phase composition or sequentially from discrete compositions.

An oral care kit can include the first composition comprising tin and/or fluoride and the second composition comprising dicarboxylic acid. The oral care kit can also include instructions directing a user to apply the first composition to an oral cavity of the user followed by applying the second composition to the oral cavity of the user. The first composition can be expectorated prior to the application of the second composition or the second composition can be applied prior to the expectoration of the first composition from the oral cavity.

The entire oral care regimen can have a duration of from one minute to about three minutes with each application step having a duration of from about 30 seconds to about 2 minutes or about 1 minute.

The components can be delivered to the oral cavity simultaneously or sequentially. The simplest case is simultaneous, continuous delivery of equal amounts of the two components or a constant ratio of the components during a single oral care session. The two components may be provided separately, such as in a dual-phase composition in two separate compositions, and then delivered simultaneously to the oral cavity. Brushing duration is sufficiently short so that the components will not be inactivated. Another use for simultaneous, continuous delivery is systems that include two components that react relatively slowly, and that will remain in the oral cavity after brushing to be absorbed by the teeth and or gums.

In the case of sequential delivery, both components may be delivered during a single oral care session, e.g., a single brushing session or other single treatment session (single use, start to finish, by a particular user, typically about 0.1 to 5 minutes), or alternatively the components may be delivered individually over multiple oral care sessions. Many combinations are possible, for example delivery of both components during a first oral care session and delivery of only one of the components during a second oral care session.

Sequential delivery during a single oral care session may take various forms. In one case, two components are delivered in alternation, as either a few relatively long duration cycles during brushing (A B A B), or many rapid-fire alternations (A B A B A B A B A B . . . A B).

In another case, two or more components are delivered one after the other during a single oral care session, with no subsequent alternating delivery in that oral care session (A followed by B). For example, a first composition comprising fluoride and/or tin can be delivered initially, to initiate brushing and provide cleansing, followed by a second composition comprising dicarboxylic acid.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Tin Stability

### Oral Care Compositions

TABLE 1
Oral Care Compositions

| Ingredient (wt %) | Ex. 1 | Ex. 2 | Ex. 3 | Ex. 4 |
|---|---|---|---|---|
| Sorbitol | 45.0000 | 47.0000 | 48.0000 | 48.0000 |
| Treated Water | 19.1091 | 20.2620 | 19.6550 | 21.1311 |
| SnF₂ | 0.4540 | — | — | 0.4540 |
| SnCl₂ (10% silica blend) | 0.5619 | — | — | 0.5619 |
| NaF | — | 0.2430 | — | — |
| Sodium Gluconate | 1.3000 | — | — | 1.3000 |
| NaOH 50% | 0.1500 | — | — | 0.8700 |
| Saccharin | 0.3500 | 0.3500 | 0.3500 | 0.4000 |
| Sucralose | 0.0800 | 0.0800 | 0.0800 | 0.2000 |
| Xanthan Gum | 0.8750 | 0.8750 | 0.8750 | 0.8750 |
| Carrageenan | 1.5000 | 1.5000 | 1.5000 | 1.5000 |
| Citric Acid | — | 0.2750 | 0.1250 | — |
| Zinc Citrate | — | — | — | 0.5330 |
| Na Citrate | 1.2050 | — | — | — |
| Potassium oxalate | 3.1400 | 3.1400 | 3.1400 | — |
| monohydrate | | | | |
| TiO₂ | 0.5000 | 0.5000 | 0.5000 | 0.5000 |
| Silica | 17.5000 | 17.5000 | 17.5000 | 17.5000 |
| Sodium Lauryl Sulfate | 7.0000 | 7.0000 | 7.0000 | 5.0000 |
| (28 wt % solution) | | | | |
| Flavor | 1.2750 | 1.2750 | 1.2750 | 1.1750 |

The treatment compositions included those from TABLE 1 and the summary TABLE 2. Ex. 1 included stannous fluoride, stannous chloride, and potassium oxalate (a dicarboxylic acid). Ex. 2 was similar to Ex. 1 except Ex. 2 replaced stannous fluoride/stannous chloride with sodium fluoride. Ex. 3 removes sodium fluoride from Ex. 2. Ex. 4 is the same as Ex. 1, but without potassium oxalate.

### Erosion Cycling Method

The enamel loss observed during erosion cycling according to TABLE 2 was determined by an in vitro model that evaluated the relative ability of oral care compositions to protect tooth surfaces against both the initiation and progression of erosive acid challenges. This model is correlated to predict clinical outcomes in an in-situ model. Briefly, tooth specimens, in groups of five per test, were cycled through 20 treatment cycles over 5 days (4 per day). Each treatment cycled progressed according to the following:
1) Two-minute exposure to a toothpaste slurry;
2) Rinsing with copious amounts of deionized water;
3) 60-minute exposure to freshly collected, whole, human saliva;
4) 10-minute erosive challenge in a solution of 1% (w/q) citric acid;
5) Rinsing with copious amounts of deionized water;
6) 60-minute exposure to freshly collected, whole, human saliva.
   Samples were stored in human saliva in a refrigerator overnight.

The erosion cycling method used here is described in detail by Eversole et al., Erosion Prevention Potential of an Over-the-Counter Stabilized SnF₂ Dentifrice Compared to 5000 ppm F Prescription-Strength Products. J. Clin. Dent. 26 (2015) 44-49, which is herein incorporated by reference.

The only change to the method was an increase in the number of enamel samples and that an optical profilometer (ContourGT 3D Optical Microscope, Bruker USA, Tucson, AZ, USA) was used to measure the 3d surface topography of the eroded samples. The average eroded depth was determined by integrating the volume of the void caused by the acid erosion with respect to the uneroded, masked reference surface and dividing it by the area of the acid-exposed enamel. Analysis of 3d measurements was done in TalyMap 3D (Taylor Hobson USA, West Chicago, IL, USA).

Crest Cavity Protection (1100 ppm F as NaF, Procter & Gamble, Cincinnati, OH, USA) and Crest ProHealth Advanced Deep Clean Mint (1100 ppm F as SnF2, Procter & Gamble, Cincinnati, OH, USA) were used as the negative and positive controls respectively. The results of the test are only valid if difference in the enamel loss of the negative and positive controls is greater than 25% the value of the enamel loss of the negative control according to Formula III. The test should be repeated if this condition is not met.

$$Erosion reduction ( \% ) = ( \frac{Enamel {Loss}_{Neg Ctrl} - Enamel {Loss}_{Pos Ctrl}}{Enamel {Loss}_{Neg Ctrl}} ) \times 100 \qquad Formula III$$

TABLE 2
Enamel Loss during Erosion Cycling (in μm)

| Example | Chemistry Applied | Enamel Loss (μm) |
|---|---|---|
| 1 | SnF₂/SnCl₂ + Oxalate | 16.16 ± 2.88 |
| 2 | NaF + Oxalate | 41.76 ± 4.14 |
| 3 | Oxalate | 48.31 ± 3.35 |
| 4 | SnF₂/SnCl₂ | 28.77 ± 1.78 |
| Crest ® Cavity | NaF | 30.72 ± 6.62 |
| Protection | | |
| Crest ® ProHealth | SnF₂ | 21.95 ± 4.41 |
| Advanced Deep | | |
| Clean Mint | | |

The enamel loss results in TABLE 2 illustrate that the dicarboxylate containing paste more efficiently delivers Sn to the enamel surface resulting in improved erosion prevention. Through the optimization of the stabilizers, the efficacy of the tin-containing product was improved. Ex. 1 (tin+oxalate) showed an enamel loss of only about 16 μm while pastes without tin, such as Ex. 2 (NaF+oxalate) and Ex. 3 (Oxalate), demonstrated enamel losses of 42 μm and 48 μm, respectively. Both Ex. 2 and Ex. 3 performed worse than either control. Importantly, Ex. 4 (tin only), which was similar to Ex. 1 except for the removal of oxalate, only had an enamel loss of 29 μm. Thus, separately, tin provides a moderate erosion benefit and oxalate provides a minimal erosion benefit. Unexpectedly, combinations of dicarboxylic acid, such as oxalate, and tin provided a superior erosion benefit. While not wishing to be bound by theory, it is believed that dicarboxylic acid acted to stabilize tin ions to more effectively deliver tin ions to the surface of the tooth to provide the erosion benefit.

### Soluble Sn

This method is suitable for determination of soluble tin in oral care toothpaste or dentifrice compositions from about 5 to about 5,000 ppm Sn in the aqueous slurry supernatant. The slurry was prepared by mixing 1 part toothpaste with 3 parts water. An aliquot of slurry was acid digested, diluted, and analyzed by inductively coupled plasma optical emission spectrometry (ICP-OES) for each toothpaste measured. Results are reported here as ppm in the neat aqueous phase of the toothpaste and/or dentifrice.

Several standards and reagents were prepared prior to the beginning of the analysis. A 5% hydrochloric acid/5% Nitric acid rinse solution was prepared by transferring 100 mL each of concentrated HCl and concentrated HNO₃ using a graduated cylinder to a 2 L volumetric flask containing about 1 L of ultrapure, 18 MΩ (DI) water. The solution was swirled to mix and diluted to the mark of the graduated flask then mixed well by repeated flask inversion.

A 1000 mg/L tin and 1000 mg/L gallium standard solution were purchased (Sigma Aldrich, Merck KGaA, Darmstadt, Germany) for preparation of the standard solutions according to TABLE 1. A pipet was used to transfer accurate quantities of the standards to a 50 mL volumetric flask while a graduated cylinder was used for the concentrated acids. After transfer, the volumetric flask was filled to the line with DI water and mixed well.

TABLE 3
Soluble Sn Standard Solution Compositions

| | | Conc | Conc | 1000 mg/L | 1000 mg/L |
|---|---|---|---|---|---|
| | | HNO₃ | HCL | Sn Std | Ga Std |
| Solution | | (mL) | (mL) | (mL) | (mL) |
| Cal Blank | | 2.5 | 2.5 | 0 | 0.2 |
| Cal 10 | mg/L Sn | 2.5 | 2.5 | 0.5 | 0.2 |
| LLOQ 0.5 | mg/L Sn | 2.5 | 2.5 | 0.025 | 0.2 |
| QC 5 | mg/L Sn | 2.5 | 2.5 | 0.25 | 0.2 |

Slurries were prepared by weighing 2.00 grams of sample into a tared round bottom 38 mL centrifuge tube containing 10 glass beads. The weight was recorded to a minimum of 0.001 g. Immedicably before slurrying, 6.0 mL of DI water was transferred to the tubes. Tubes were capped and placed on a vortexer, mixing the samples for 60 minutes at 1200 rpm. The tubes were removed from the vortexer immediately following completion of the mixing cycle and placed in a centrifuge. They were centrifuged at 21,000 relative centrifugal force (RCF) for 10 minutes. Immediately following completion of centrifugation, the tubes were removed, and the supernatant was gently mixed by inverting slowly three times making sure the solid plug at the bottom of the centrifuge tube was not disturbed before the sample was decanted. The supernatant was then decanted into a15 mL screw cap sample tube, making sure most of the supernatant was transferred.

The supernatant samples were then digested by accurately weighing (to 0.001 g) a 0.5 mL aliquot of supernatant into a 50 mL Falcon tube. Then 2.5 mL of concentrated HCl and HNO3 were added. The tubes were covered with a polypropylene watch glass and placed in a preheated block digester at 90° C. for 30 minutes. The samples were removed the from the heat, the watch class was rinsed three times with DI water (with about 1 mL each time), and that rinsate was added to the digested supernatant. The gallium standard (0.2 mL) was pipetted into the digested supernatant and then the supernatant samples were diluted to 50 mL with DI water. The tubes were capped and mixed. A digestion method blank was prepared in the same manner using 0.5 mL of DI water instead of supernatant. A method blank was prepared and analyzed for each set of hot block digestions if more samples were prepared than could fit into the hot block at once.

The ICP-OES (Perkin-Elmer 8300, Waltham, MA, USA) was operated by a trained and qualified operator with demonstrated capability of running the instrument and accurately determining the quantity of tin in oral care compositions. The ICP-OES operation parameters were selected based on the model and configuration according to the manufacturer's instructions. Samples were analyzed according to the following protocol:
1. The ICP-OES was preheated and optimized according to the manufacturer's guidelines. Recommended system checks were performed. The system was conditioned for 30 minutes prior to analysis by running the HCl/HNO₃ rinse solution through the sample introduction system.
2. The method for determining tin using a gallium internal standard at the manufacturer recommended wavelengths, integration times, and observation modes was loaded into the operating computer.
3. The 5% HCl/5% HNO₃ rinse solution was used to rinse the sample introduction system between the analysis of each blank, standard, or test solution.
4. Three to five readings were recorded for all solutions during analysis.
5. The calibration blank was analyzed.
6. The 10 ppm Sn standard was measured.
7. The 5 ppm Sn standard was measured.
8. The 0.5 ppm LLOQ tin standard was measured.
9. The method blank was measured.
10. The test solutions were measured.
11. The 5 ppm Sn standard was re-measured after every sixth test solution and after the last sample. Enough standard was made to complete the analysis.
12. The 0.5 ppm LLOQ tin standard was measured at the end of the sample analysis.

The analysis was considered successful if the % relative standard deviation of the replicate readings for the 10 ppm and the 5 ppm tin standards was less than about 3%. The 5-ppm check standard was within 96-104% of its value. The LLOQ was within 75-125% of its value. The method blank showed less tin signal intensity than the LLOQ sample. The recovery of the internal standard in each analyzed solution was within 90-130% of its value.

The soluble tin was determined according to the following formula:

$$Soluble Tin in Composition = \frac{Sn from ICP ( \frac{μ g}{mL} ) \times Final volume of test solution ( mL )}{Supernatant Weight ( g )} \times \frac{Soluble Mass of Composition ( g ) + Slurry Water ( g )}{Total Composition Mass ( g )} \qquad Formula IV$$

TABLE 4
Soluble Sn (ppm)

| Example | Chemistry Applied | ~22 d | ~62 d | ~121 d | ~212 d |
|---|---|---|---|---|---|
| 1 | SnF₂/SnCl₂ + Oxalate | 4811 | 4772 | 4352 | 4238 |
| 4 | SnF₂/SnCl₂ | 2424 | 1974 | 1630 | 1730 |

TABLE 4 shows soluble Sn measurements for Ex. 1 (tin+oxalate) and Ex. 4 (tin only). Unexpectedly, Ex. 1 (tin+oxalate) displayed more than double the amount of soluble Sn than Ex. 4 (tin only) after 22 days, 62 days, 121 days, and 212 days. Thus, dicarboxylic acid, such as oxalate, can act to deliver higher amounts of tin ions to the oral cavity.

### Fluoride Stability

### Method for Measuring Soluble Fluoride Ion in Compositions Containing MFP

This method provides a measure of the concentration of all soluble fluoride ions in diluted MFP dentifrice solutions. The dentifrice was diluted in accordance with industry standards and the preamble to the FDA “Anticaries Drug Products for Over-the-Counter Human Use” monograph. A slurry was prepared by thoroughly dispersing/mixing the diluted dentifrice. The ion selective electrode (ISE) (e.g., Orion 9609BNWP combination, Mettler Toledo PerfectION, Cole Parmer Combination EW-27504-14) was calibrated using fluoride ion standard solutions such that the results were within 2.0% of the known μg/g value of the standard. Each final sample solution was then analyzed for the concentration of soluble fluoride ions by automated standard addition with the ion selective electrode.

For MFP-containing compositions, samples of the MFP-containing compositions were prepared by weighing 2.99-3.01 g g of the composition into a plastic specimen cup, adding 29.9-30.1 g of DI water, and stirring vigorously on a magnetic stir plate for 10 minutes so that the slurry was homogeneous. The slurry was centrifuged (e.g., Sorvall RC28S with SS-34 rotor) for a minimum of 10 minutes at 15000 RCF (or higher) at 20-25° C. A specified volume of the resulting supernatant was acid hydrolyzed, neutralized, and diluted with water and TISAB II. Specifically, 4.99-5.01 g of the supernatant was poured into another plastic specimen cup, and 9.95-10.05 g of 2N HCl Solution was mixed into the supernatant. The samples were stored at about 60.0° C. for 20 minutes and then cooled at room temperature for at least 20 minutes. Then, 10.2-10.3 g of 2N NaOH Solution was mixed into the cooled samples. A 10 mL aliquot of each final sample solution or calibration standard was then diluted with 10 ml of DI water, 20 mL of TISAB II buffer, and then analyzed for the concentration of soluble fluoride ions by the ion selective electrode. Because the calibration standards and final samples are diluted identically with water and TISAB II, there is no correction needed for this step in the calculation of soluble fluoride in the product.

The soluble fluoride in MFP-containing compositions was determined according to the following formula:

$$Soluble F in Product ( \frac{μ g}{g} ) = Dilution Factor \times \frac{Supernatant wt ( g ) + 2 N HC 1 wt ( g ) + 2 N NaOH wt ( g )}{Supernatant wt ( g )} \times F sample ( \frac{μ g}{g} )$$

where the dilution factor is calculated according to the following formula:

$$Dilution Factor = 11 - ( \frac{\% of insoluble components of toothpaste}{100} ) \qquad Formula V$$

The insoluble components of a toothpaste include those things that do not dissolve in water, such as silica, titanium dioxide, mica, etc.

### Method for Measuring Soluble Fluoride Ion in Compositions Containing NaF or SnF₂

This method provides a measure of the concentration of all soluble fluoride ions in diluted NaF or SnF₂ dentifrice solutions. The dentifrice was diluted in accordance with industry standards and the preamble to the FDA “Anticaries Drug Products for Over-the-Counter Human Use” monograph. A slurry was prepared by thoroughly dispersing/mixing the diluted dentifrice. The ion selective electrode (ISE) (e.g., Orion 9609BNWP combination, Mettler Toledo PerfectION, Cole Parmer Combination EW-27504-14) was calibrated using fluoride ion standard solutions such that the results were within 2.0% of the known μg/g value of the standard. Each final sample solution was then analyzed for the concentration of soluble fluoride ions by automated standard addition with the ion selective electrode.

For compositions containing NaF without tin, 5.99-6.01 g of the composition was added to a plastic specimen cup with 17.97-18.03 g of DI water. For compositions containing SnF₂ or NaF in combination with SnF₂ or SnCl₂, 2.99-3.01 g of the composition was added to a plastic specimen cup with 29.9-30.1 g of DI water. The mixtures were stirred vigorously on a magnetic stir plate for 10 minutes so that the slurry was homogeneous. The stirred samples were shaken (e.g., MaxQ Mini 4450Shaker, Thermo Scientific Model #SHKE 4450, Cole-Parmer P/N K51960-04) for 60 minutes at a temperature of about 37.0° C. The slurries were then centrifuged within 10 minutes for a minimum of 10 minutes at 15,000 RCF or higher at about 20 to 25° C. A 5 mL aliquot of each final sample solution or calibration standard was then diluted with 10 ml of DI water, 20 mL of TISAB II buffer, and then was analyzed for the concentration of soluble fluoride ions by the ion selective electrode. Because the calibration standards and final samples are diluted identically with water and TISAB II, there is no correction needed for this step in the calculation of soluble fluoride in the product.

The soluble fluoride in NaF- and Sn-containing compositions was determined according to the following formula:

Soluble F in Product (μg/g)=Dilution Factor×F sample (μg/g)

where the dilution factor is calculated according to the following formula:

$$Dilution Factor ( NaF ) = 4 - ( \frac{\% of insoluble components of toothpaste}{100} ) Dilution Factor ( Sn - Containing ) = 11 - ( \frac{\% of insoluble components of toothpaste}{100} ) \qquad Formula VI$$

The insoluble components of a toothpaste include those things that do not dissolve in water, such as silica, titanium dioxide, mica, etc.

### pH Measurement Method

The classical definition of pH is −log[H+ (aq.)]. For all practical purposes, it is the value given by a properly calibrated potentiometric instrument which uses an indicator electrode (sensitive to the hydrogen ion (H+) activity) and a suitable reference electrode. The instrument/electrode should be capable of performing a 2-point pH calibration and have resolution to at least 0.01 pH with temperature compensation (resolution to at least 0.1° C.) to convert the millivolt signal to pH units at any temperature automatically or with manual entry of temperature into the measurement system. In this particular method, the apparent hydrogen-ion concentration or pH is measured for aqueous solutions of products using a potentiometric instrument and a suitable electrode at room temperature (typically 23-27° C.) using an Automatic Temperature Compensating probe.

The pH Meter should be a meter capable of reading to at least 0.01 pH units, such as Mettler Toledo Seven Excellence pH meter S400.

The Basic pH Electrode may be a combined liquid filled electrode with a sleeved diaphragm, such as Orion Ross Sure Flow combination: Orion #8172BNWP. Before measurements were taken, the meter was calibrated using two calibration buffers of different pH such that the pH slope was 90-105% and the offset was ±30 mV (0.5 pH units at 25.0° C.). After calibration, a verification buffer was analyzed. The pH of the verification buffer was between the two calibration buffers was not the same as either of the calibration buffers. The measured pH of the verification buffer was within +0.05 units of the labeled value.

To prepare the samples, a 1 part product to 3 parts water (by weight) slurry was made. The slurry must be homogeneous without lumps and/or product remaining on the container walls. A sample size of 5 g to 12 g of product should be used to ensure a representative sample is obtained. The sample should be measured following at least 10 minutes of stirring.

A suitable quantity of the sample was added to a container at room temperature. Enough solution was used to cover the electrode tip completely. The pH of samples was recorded after one minute. If the meter could not force a reading at one minute, the reading was taken when it stabilized. After each measurement, the electrode was rinsed with deionized water.

### Preparation of Oral Care Compositions

The oral care compositions of TABLE 5 were prepared by combining one or more humectants, water, sweetener(s), and a portion of flavor to create a liquid mixture. The salts (citrate, gluconate, fluoride sources, stannous sources, dicarboxylic acid (DCA), etc. where applicable) were added to the mixture. The liquid mixture was mixed, recirculated, and homogenized at 25° C. until homogeneous and salts were completely dissolved. Next, sodium hydroxide (50% solution) was added to the liquid mixture where applicable, and the liquid mixture was homogenized at 25° C. until homogeneous. A separate powder mixture was prepared by combining the thickening silica and opacifier with any thickening agents, such as xanthan gum, carrageenan, and/or sodium carboxymethylcellulose. The powder mixture was then combined with the liquid mixture and mixed, recirculated, and homogenized completely until all powders are fully wetted and the texture was smooth. Then the abrasive silica was added to the mixture and mixed until all silica was fully hydrated. Next, the surfactant, such as sodium lauryl sulfate, and the remaining flavor were added to the mixture. The contents were mixed, recirculated, and homogenized at 25° C. under vacuum until homogeneous and entrained air was removed by the vacuum.

TABLE 5
Oral Care Compositions

| Ingredient (wt. %) | Ex 5 | Ex. 6 | Ex. 7 | Ex. 8 | Ex. 9 | Ex. 10 | Ex. 11 |
|---|---|---|---|---|---|---|---|
| Sorbitol (70% soln) | 60.000 | 62.000 | 61.000 | 63.000 | 59.000 | 61.000 | 65.000 |
| Water | 8.770 | 7.585 | 8.667 | 7.482 | 8.924 | 6.696 | 8.167 |
| NaF | — | — | 0.243 | 0.243 | 0.243 | 0.243 | 0.243 |
| MFP | 1.140 | 1.140 | — | — | — | — | — |
| Sodium Gluconate | — | — | — | — | 1.300 | 1.300 | — |
| NaOH (50% soln) | — | 1.350 | — | 1.350 | 0.208 | 1.681 | — |
| Saccharin | 0.400 | 0.400 | 0.400 | 0.400 | 0.400 | 0.400 | 0.400 |
| Xanthan Gum | 0.500 | 0.500 | 0.500 | 0.500 | 0.500 | 0.500 | 0.500 |
| Carboxymethyl | 1.000 | 1.000 | 1.000 | 1.000 | 1.000 | 1.000 | 1.000 |
| Cellulose | | | | | | | |
| Citric Acid | 0.800 | — | 0.800 | — | 1.035 | 0.805 | 0.440 |
| Sodium Citrate | — | — | — | — | — | 0.350 | — |
| Potassium Oxalate | 3.140 | — | 3.140 | — | 3.140 | — | — |
| Malonic Acid | — | 1.775 | — | 1.775 | — | 1.775 | — |
| TiO₂ | 0.500 | 0.500 | 0.500 | 0.500 | 0.500 | 0.500 | 0.500 |
| Thickening Silica | 0.500 | 0.500 | 0.500 | 0.500 | 0.500 | 0.500 | 0.500 |
| Silica | 17.000 | 17.000 | 17.000 | 17.000 | 17.000 | 17.000 | 17.000 |
| Sodium Lauryl | 5.000 | 5.000 | 5.000 | 5.000 | 5.000 | 5.000 | 5.000 |
| Sulfate (29% soln) | | | | | | | |
| Flavor | 1.250 | 1.250 | 1.250 | 1.250 | 1.250 | 1.250 | 1.250 |

The oral care compositions of TABLE 6 and TABLE 7 were prepared following the procedure described previously.

TABLE 6
Oral Care Compositions

| Ingredient | | | | | | | | | |
|---|---|---|---|---|---|---|---|---|---|
| (wt %) | Ex. 12 | Ex. 13 | Ex. 14 | Ex. 15 | Ex. 16 | Ex. 17 | Ex. 18 | Ex. 19 | Ex. 20 |
| Sorbitol | 83.000 | 84.000 | 39.000 | 29.000 | 31.000 | 62.000 | 35.000 | 35.000 | 66.000 |
| (70% soln) | | | | | | | | | |
| Glycerin | — | — | 10.000 | 10.000 | 10.000 | 10.000 | 10.000 | 10.000 | 10.000 |
| Water | 2.182 | 0.902 | 15.320 | 17.840 | 17.440 | 8.440 | 15.685 | 15.310 | 6.310 |
| NaF | 0.243 | 0.243 | — | — | — | — | — | — | — |
| MFP | — | — | 1.140 | 1.140 | 1.140 | 1.140 | 1.140 | 1.140 | 1.140 |
| Sodium | 1.300 | 1.300 | — | — | — | — | — | — | — |
| Gluconate | | | | | | | | | |
| NaOH | 0.450 | 1.975 | — | — | — | — | 1.650 | 2.750 | 2.750 |
| (50% soln) | | | | | | | | | |
| Saccharin | 0.400 | 0.400 | 0.500 | 0.500 | 0.500 | 0.500 | 0.500 | 0.500 | 0.500 |
| Sucralose | — | — | 0.200 | 0.200 | 0.200 | 0.200 | 0.200 | 0.200 | 0.200 |
| (25% soln) | | | | | | | | | |
| Xanthan Gum | 0.500 | 0.500 | 0.350 | 0.350 | 0.350 | 0.350 | 0.350 | 0.350 | 0.350 |
| Carboxymethyl | 1.000 | 1.000 | — | — | — | — | — | — | — |
| Cellulose | | | | | | | | | |
| Carrageenan | — | — | 0.700 | 0.700 | 0.700 | 0.700 | 0.700 | 0.700 | 0.700 |
| (10% silica | | | | | | | | | |
| blend) | | | | | | | | | |
| Citric Acid | 1.035 | 0.805 | 0.800 | 3.300 | 1.815 | 1.815 | 1.650 | 0.650 | 0.650 |
| Sodium Citrate | — | 0.350 | — | 4.980 | 1.725 | 1.725 | 2.500 | 1.000 | 1.000 |
| Potassium | 3.140 | — | 3.140 | 3.140 | 6.280 | 6.280 | — | — | — |
| Oxalate | | | | | | | | | |
| Malonic Acid | — | 1.775 | — | — | — | — | 1.775 | 3.550 | 3.550 |
| TiO₂ | 0.500 | 0.500 | 0.500 | 0.500 | 0.500 | 0.500 | 0.500 | 0.500 | 0.500 |
| Thickening | — | — | 5.000 | 5.000 | 5.000 | — | 5.000 | 5.000 | — |
| Silica | | | | | | | | | |
| Silica | — | — | 17.000 | 17.000 | 17.000 | — | 17.000 | 17.000 | — |
| Sodium Lauryl | 5.000 | 5.000 | 5.000 | 5.000 | 5.000 | 5.000 | 5.000 | 5.000 | 5.000 |
| Sulfate | | | | | | | | | |
| (29% soln) | | | | | | | | | |
| Flavor | 1.250 | 1.250 | 1.350 | 1.350 | 1.350 | 1.350 | 1.350 | 1.350 | 1.350 |

TABLE 7
Oral Care Compositions.

| Ingredient | | | | | | | | | |
|---|---|---|---|---|---|---|---|---|---|
| (wt %) | Ex. 21 | Ex. 22 | Ex. 23 | Ex. 24 | Ex. 25 | Ex. 26 | Ex. 27 | Ex. 28 | Ex. 29 |
| Sorbitol | 38.000 | 34.000 | 28.000 | 60.000 | 30.000 | 61.000 | 29.000 | 31.000 | 62.000 |
| (70% soln) | | | | | | | | | |
| Glycerin | 10.000 | 10.000 | 10.000 | 10.000 | 10.000 | 10.000 | 10.000 | 10.000 | 10.000 |
| Water | 17.159 | 18.509 | 17.844 | 7.844 | 17.079 | 8.079 | 17.309 | 15.909 | 6.909 |
| SnF₂ | 0.454 | 0.454 | 0.454 | 0.454 | 0.454 | 0.454 | 0.454 | 0.454 | 0.454 |
| SnCl₂ (10% | 0.562 | 0.562 | 0.562 | 0.562 | 0.562 | 0.562 | 0.562 | 0.562 | 0.562 |
| silica blend) | | | | | | | | | |
| Sodium | 1.300 | 1.300 | 1.300 | 1.300 | 1.300 | 1.300 | 1.300 | 1.300 | 1.300 |
| Gluconate | | | | | | | | | |
| NaOH (50% | 0.750 | 0.250 | — | — | — | — | — | 2.000 | 2.000 |
| soln) | | | | | | | | | |
| Saccharin | 0.500 | 0.500 | 0.500 | 0.500 | 0.500 | 0.500 | 0.500 | 0.500 | 0.500 |
| Xanthan Gum | 0.350 | 0.350 | 0.350 | 0.350 | 0.350 | 0.350 | 0.350 | 0.350 | 0.350 |
| Carrageenan | 0.700 | 0.700 | 0.700 | 0.700 | 0.700 | 0.700 | 0.700 | 0.700 | 0.700 |
| (10% silica | | | | | | | | | |
| blend) | | | | | | | | | |
| Citric Acid | — | — | 2.900 | 2.900 | 1.700 | 1.700 | 1.200 | 0.000 | — |
| Sodium | 1.550 | 1.560 | 5.575 | 5.575 | 2.400 | 2.400 | 8.175 | 5.000 | 5.000 |
| Citrate | | | | | | | | | |
| Potassium | | 3.140 | 3.140 | 3.140 | 6.280 | 6.280 | — | — | — |
| Oxalate | | | | | | | | | |
| Malonic Acid | — | — | — | — | — | — | 1.775 | 3.550 | 3.550 |
| TiO₂ | 0.500 | 0.500 | 0.500 | 0.500 | 0.500 | 0.500 | 0.500 | 0.500 | 0.500 |
| Thickening | 5.000 | 5.000 | 5.000 | — | 5.000 | — | 5.000 | 5.000 | — |
| Silica | | | | | | | | | |
| Silica | 17.000 | 17.000 | 17.000 | — | 17.000 | — | 17.000 | 17.000 | — |
| Sodium | 5.000 | 5.000 | 5.000 | 5.000 | 5.000 | 5.000 | 5.000 | 5.000 | 5.000 |
| Lauryl | | | | | | | | | |
| Sulfate | | | | | | | | | |
| (29% soln) | | | | | | | | | |
| Flavor | 1.175 | 1.175 | 1.175 | 1.175 | 1.175 | 1.175 | 1.175 | 1.175 | 1.175 |

### Soluble Fluoride

The fluoride solubility was measured over time for Examples 1, 2, and 5-11. TABLE 8 shows the percentage of soluble fluoride remaining after storage at ambient conditions where the temperature is about 20° C. The measurements included are those taken the closest to 1, 3, 6, or 12 months. Of note, some of the data indicates a percentage of soluble fluoride over 100%. This is an artifact of variability in the method, which may be +/−2%.

TABLE 8
Remaining Soluble Fluoride (% of theoretical F)

| | F | Added F | | | | | |
|---|---|---|---|---|---|---|---|
| | Source | (ppm) | DCA | 1 mo. | 3 mo. | 6 mo. | 12 mo. |
| Ex. 5 | MFP | 1500 | Oxalate | 90 | 81 | 84 | 88 |
| Ex. 6 | MFP | 1500 | Malonate | 90 | 86 | 86 | 90 |
| Ex. 7 | NaF | 1100 | Oxalate | 48 | 46 | 45 | 45 |
| Ex. 8 | NaF | 1100 | Malonate | 82 | 76 | 74 | 71 |
| Ex. 9 | NaF | 1100 | Oxalate | 55 | 51 | 51 | 50 |
| Ex. 10 | NaF | 1100 | Malonate | 79 | 74 | 73 | 65 |
| Ex. 11 | NaF | 1100 | None | 82 | 77 | 75 | 72 |
| Ex. 1 | SnF₂ | 1100 | Oxalate | 88 | — | 102 | — |
| Ex. 2 | NaF | 1100 | Oxalate | 65 | — | 69 | — |

Examples 2 and 7-11 contained NaF. In NaF-containing compositions, it is believed that the presence of silica is a driver of soluble fluoride instability. At low pH conditions, fluorosilicates can form as can be inferred from low F-stability in Ex. 11, which has NaF at low pH and no DCA, but the stability of Ex. 11 is better than when a DCA is added, especially oxalate like in Ex. 7. Without wishing to be bound by theory, it is believed that the oxalate is extracting metals from the silica further reducing the F-stability by forming metal fluoride complexes. Those complexes are either insoluble or reduce the availability of fluoride anion to be measured, or both. The abrasive silica used to make these compositions contain aluminum, calcium, and iron as impurities. A batch of the abrasive silica was measured to have about 1300 ppm total Al, about 60 ppm total Ca, and about 250 ppm total Fe. Oxalate may extract some of these metal impurities contained in the silica facilitating formation of the aforementioned metal fluoride species. The addition of sodium gluconate or sodium citrate in compositions containing NaF and either oxalate (Ex. 9) and malonate (Ex. 10) did not provide a noticeable chelant effect on the liberated metals to prevent their interaction with fluoride. Based on these results, gluconate or citrate did not affect the fluoride stability in a composition with NaF and silica at low pH.

The results in TABLE 8 illustrate the fluoride stability over the course of one year at ambient conditions relative to the formulated fluoride source, level, and dicarboxylate source. The fluoride source/level/DCA combination was considered stable if it maintained more than 50% of its formulated value at 12 months. The fluoride source, MFP, provided a stable fluoride value in the presence of both oxalate (Ex. 5) and malonate (Ex. 6). The fluoride source, NaF, provided a stable fluoride value in the presence of malonate (Ex. 8 and Ex. 10) or with no DCA (Ex. 11). NaF was not stable in combination with oxalate (Ex. 7 and Ex. 9)—although the amount was steady over time, the level of soluble fluoride was unacceptably low in view of the amount of added F. However, the fluoride source, SnF₂, also provided a stable fluoride value in the presence of oxalate (Ex. 1) at 6 months that was higher than the value for NaF/oxalate at 6 months (Ex. 2). It is worth noting that the pH of Examples 1 and 2 was comparatively higher than the pH of Examples 5-11. This may explain the directionally better stability of Ex. 2 compared to Examples 7 and 9, which all contain NaF and oxalate. The significantly better stability observed with SnF₂ is not explained by higher pH alone as the aged pH of Examples 1 and 2 is comparable. While not wishing to be bound by theory, it is believed that because of oxalate's high affinity for metals, it facilitated the interaction between residual metals from the silica and the soluble fluoride ion. However, without wishing to be bound by theory, the combination of Sn with oxalate was believed to have formed a Sn-oxalate-F complex in the formulation preventing the association of fluoride with residual metals from the silica. This soluble fluoride Sn—F complex is then easily recovered during fluoride measurements.

TABLE 9 shows the percentage of soluble fluoride remaining after storage at 40° C. and 75% relative humidity conditions. The measurements included are those taken initially and then closest to 1, 2, 3 and 6 months. Aging at accelerated conditions (i.e., higher temperature) was performed for Examples 12 to 29 compared to Examples 1, 2, and 5-11. In general, 3 months at 40° C. and 75% RH is considered to be equivalent to 12 months at ambient (20° C.), and 6 months at 40° C. and 75% RH is considered to be equivalent to 24 months at ambient.

TABLE 9
Remaining Soluble Fluoride (% of theoretical F)

| | F | Added F | | | | | | | |
|---|---|---|---|---|---|---|---|---|---|
| | Source | (ppm) | DCA | Silica | Initial | 1 mo. | 2 mo. | 3 mo. | 6 mo. |
| Ex. 12 | NaF | 1100 | Oxalate | no | 91 | 101 | 101 | 101 | 99 |
| Ex. 13 | NaF | 1100 | Malonate | no | 92 | 101 | 100 | 100 | 99 |
| Ex. 14 | MFP | 1500 | Oxalate | yes | 93 | 91 | 89 | 86 | 84 |
| Ex. 15 | MFP | 1500 | Oxalate | yes | 94 | 91 | 88 | 84 | 81 |
| Ex. 16 | MFP | 1500 | Oxalate | yes | 92 | 90 | 85 | 82 | 74 |
| Ex. 17 | MFP | 1500 | Oxalate | no | 94 | 96 | 92 | 91 | 81 |
| Ex. 18 | MFP | 1500 | Malonate | yes | 95 | 93 | 91 | 89 | 87 |
| Ex. 19 | MFP | 1500 | Malonate | yes | 95 | 93 | 90 | 88 | 88 |
| Ex. 20 | MFP | 1500 | Malonate | no | 93 | 98 | 96 | 97 | 93 |
| Ex. 21 | SnF₂ | 1100 | None | yes | 97 | 95 | 95 | 88 | 89 |
| Ex. 22 | SnF₂ | 1100 | Oxalate | yes | 91 | 84 | 93 | 82 | 84 |
| Ex. 23 | SnF₂ | 1100 | Oxalate | yes | 91 | 85 | 82 | 80 | 78 |
| Ex. 24 | SnF₂ | 1100 | Oxalate | no | 100 | 87 | 97 | 96 | 96 |
| Ex. 25 | SnF₂ | 1100 | Oxalate | yes | 60 | 50 | 47 | 51 | 79 |
| Ex. 26 | SnF₂ | 1100 | Oxalate | no | 68 | 71 | 63 | 70 | 64 |
| Ex. 27 | SnF₂ | 1100 | Malonate | yes | 90 | 81 | 80 | 77 | 74 |
| Ex. 28 | SnF₂ | 1100 | Malonate | yes | 90 | 83 | 80 | 78 | 77 |
| Ex. 29 | SnF₂ | 1100 | Malonate | no | 100 | 99 | 99 | 98 | 98 |

Examples 12 and 13 contain NaF. Examples 12 and 13 are versions of Examples 9 and 10, but without silica present in the composition and with an adjustment to the levels of sorbitol and water. It can be seen in TABLE 10 that removal of silica in Examples 12 and 13 unexpectedly and significantly improves soluble Fluoride stability for NaF compositions at low pH and with Oxalate or Malonate compared to Examples 9 and 10 in TABLE 6.

Examples 14-20 contain MFP. Examples 14, 15, and 16 contain MFP and Oxalate at varying levels. Ex. 17 is the same composition as Ex. 16 except that silica is not present and is replaced with an adjustment to the levels of sorbitol and water. Examples 18 and 19 contain MFP and Malonate at varying levels. Ex. 20 is the same composition as Ex. 19 except that silica is not present and is replaced with an adjustment to the levels of sorbitol and water. As can be seen in TABLE 10, with MFP there 10 is good soluble Fluoride stability and removal of silica has minimal if any impact.

Examples 21-29 contain SnF₂. Ex. 21 is a SnF2 containing composition at around pH 6 which does not include a DCA. It demonstrates good soluble Fluoride stability. Examples 22 and 23 add Oxalate and reduce the pH to 5.5 and 4.6, respectively-these examples demonstrate good soluble Fluoride stability, although not nearly as good as Ex. 21. Ex. 24 is the same composition as Ex. 23 except that silica is not present and is replaced with an adjustment to the levels of sorbitol and water. While Examples 22 and 23 show good soluble Fluoride stability, Ex. 24 demonstrates significantly improved soluble Fluoride stability—even better than Ex. 21.

Examples 25 and 26 contain SnF₂ and Oxalate like Examples 23 and 24, but with twice the level of Oxalate. Ex. 25 contains silica and Ex. 26 is the same composition as Ex. 25 except that silica is not present and is replaced with an adjustment to the levels of sorbitol and water. In both examples soluble Fluoride stability is good, but removing silica did not improve soluble Fluoride stability as was seen at lower Oxalate levels. While not wishing to be bound by theory, it is believed that formation of Sn-Oxalate-Fluoride species increases and possibly exceeds the solubility limit of these species and reduces Fluoride availability to be measured.

Examples 27 and 28 are SnF2 with Malonate at increasing malonate levels, both demonstrate good Fluoride stability, however Ex. 29, which is the same composition as Ex. 28 except that silica is not present and is replaced with an adjustment to the levels of sorbitol and water, demonstrated significantly improved soluble Fluoride stability.

TABLE 10 shows the change in pH while Examples 1, 2, and 5-11 were stored at ambient conditions where the temperature is about 20° C. The measurements included are those taken initially and then closest to 1, 3, 6, or 12 months.

TABLE 10
pH Stability over Time

| | F | | | | | | |
|---|---|---|---|---|---|---|---|
| | Source | DCA | Initial | 1 mo. | 3 mo. | 6 mo. | 12 mo. |
| Ex. 5 | MFP | Oxalate | 4.70 | 4.64 | 4.64 | 4.73 | 4.75 |
| Ex. 6 | MFP | Malonate | 4.81 | 4.81 | 4.86 | 4.90 | 4.91 |
| Ex. 7 | NaF | Oxalate | 4.70 | 4.62 | 4.70 | 4.73 | 4.72 |
| Ex. 8 | NaF | Malonate | 4.76 | 4.76 | 4.85 | 4.88 | 4.87 |
| Ex. 9 | NaF | Oxalate | 4.77 | 4.74 | 4.79 | 4.84 | 4.83 |
| Ex. 10 | NaF | Malonate | 4.77 | 4.66 | 4.73 | 4.76 | 4.75 |
| Ex. 11 | NaF | None | 4.82 | 4.75 | 4.86 | 4.90 | 4.86 |
| Ex. 1 | SnF₂ | Oxalate | 6.01 | 5.74 | 5.71 | 5.68 | 5.64 |
| Ex. 2 | NaF | Oxalate | 5.31 | 5.47 | 5.46 | 5.56 | 5.52 |

The results in TABLE 11 illustrate the change in pH during storage of Examples 12-27 at 40° C. 75% relative humidity conditions. The measurements included are those taken initially and then closest to 1, 2, 3 and 6 months.

TABLE 11
pH Stability over Time

| | F | | | | | | | |
|---|---|---|---|---|---|---|---|---|
| | Source | DCA | Silica | Initial | 1 mo. | 2 mo. | 3 mo. | 6 mo. |
| Ex. 12 | NaF | Oxalate | no | 4.73 | 4.70 | 4.74 | 4.71 | 4.73 |
| Ex. 13 | NaF | Malonate | no | 4.78 | 4.79 | 4.81 | 4.81 | 4.86 |
| Ex. 14 | MFP | Oxalate | yes | 4.70 | 4.74 | 4.80 | 4.78 | 4.81 |
| Ex. 15 | MFP | Oxalate | yes | 4.56 | 4.57 | 4.62 | 4.59 | 4.63 |
| Ex. 16 | MFP | Oxalate | yes | 4.64 | 4.67 | 4.73 | 4.69 | 4.74 |
| Ex. 17 | MFP | Oxalate | no | 4.62 | 4.58 | 4.61 | 4.61 | 4.62 |
| Ex. 18 | MFP | Malonate | yes | 4.66 | 4.71 | 4.75 | 4.73 | 4.77 |
| Ex. 19 | MFP | Malonate | yes | 4.54 | 4.59 | 4.64 | 4.63 | 4.67 |
| Ex. 20 | MFP | Malonate | no | 4.51 | 4.50 | 4.54 | 4.55 | 4.59 |
| Ex. 21 | SnF₂ | None | yes | 6.37 | 6.19 | 6.21 | 6.17 | 6.20 |
| Ex. 22 | SnF₂ | Oxalate | yes | 5.49 | 5.49 | 5.59 | 5.56 | 5.29 |
| Ex. 23 | SnF₂ | Oxalate | yes | 4.57 | 4.62 | 4.64 | 4.65 | 4.58 |
| Ex. 24 | SnF₂ | Oxalate | no | 4.64 | 4.58 | 4.62 | 4.63 | 4.64 |
| Ex. 25 | SnF₂ | Oxalate | yes | 4.57 | 4.69 | 4.69 | 4.73 | 4.67 |
| Ex. 26 | SnF₂ | Oxalate | no | 4.64 | 4.64 | 4.67 | 4.66 | 4.63 |
| Ex. 27 | SnF₂ | Malonate | yes | 4.59 | 4.63 | 4.63 | 4.60 | 4.69 |
| Ex. 28 | SnF₂ | Malonate | yes | 4.68 | 4.73 | 4.74 | 4.75 | 4.78 |
| Ex. 29 | SnF₂ | Malonate | no | 4.63 | 4.68 | 4.70 | 4.71 | 4.75 |

At the end of the measurement period, 6 months, all of the examples containing DCAs had a pH of about 4.5 to about 5.5, with Examples 1 and 2 being slightly above 5.5. Ex. 21, which does not contain a DCA, had a pH between 6.1 and 6.4. In this pH range, the benefit (e.g., whitening) of the DCA can be effective while reducing the possibility of oral hard tissue damage.

These results demonstrate that formulating fluoride and a DCA in a toothpaste is a special challenge relative to other oral care compositions, e.g., rinse, because of the presence of the abrasive comprising residual metals. The low pH condition in the presence of a DCA can extract residual metal from the silica allowing it to interact with fluoride ion unless that fluoride ion is protected. That protection can be in the form of sodium monofluorophosphate where the fluoride is covalently bound to phosphorous and must be released through acid digestion or enzymatic action in the mouth. That protection can also be in the form of a complex of the fluoride ion with stannous and other stabilizing agents like gluconate, citrate, oxalate, malonate, or combinations thereof. Once stabilized, the fluoride is available to provide anticavity benefits in the oral care composition while the DCA and Sn can deliver their respective primary benefits, namely stain removal and anti-gingivitis, respectively.

The terms “substantially,” “essentially,” “about,” “approximately,” and the like, as may be used herein, represent the inherent degree of uncertainty that may be attributed to any quantitative comparison, value, measurement, or other representation. These terms also represent the degree by which a quantitative representation may vary from a stated reference without resulting in a change in the basic function of the subject matter at issue. Further, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
(a) from about 0.1% to about 15%, by weight of the composition, of a dicarboxylic acid or a salt thereof;
(b) a fluoride ion source; and
(c) silica abrasive,
wherein the composition has a pH in a range of from about 4 to about 5.5.

2. The composition of claim 1, wherein the oral care composition, after 12 months of aging at about 20° C., has a soluble fluoride level that is more than 50% of a theoretical amount of fluoride.

3. The composition of claim 1, wherein the pH is in a range of from about 4 to 5.3.

4. The composition of claim 1, wherein the pH is in a range of from about 4 to about 5.

5. The composition of claim 1, wherein the silica abrasive contains residual iron, aluminum, or calcium.

6. The composition of claim 1, wherein the silica abrasive comprises up to about 500 ppm, by weight of the silica abrasive, of iron.

7. The composition of claim 1, wherein the silica abrasive comprises up to about 2000 ppm, by weight of the silica abrasive, of aluminum.

8. The composition of claim 1, wherein the silica abrasive comprises up to about 100 ppm, by weight of the silica abrasive, of calcium.

9. The oral care composition of claim 8, wherein the fluoride ion source comprises stannous fluoride, sodium fluoride, sodium monofluorophosphate, amine fluoride, or combinations thereof.

10. The oral care composition of claim 1, wherein the dicarboxylic acid comprises oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, fumaric acid, terephthalic acid, salts thereof, or combinations thereof.

11. The oral care composition of claim 1, wherein the oral care composition comprises a monodentate ligand and a polydentate ligand.

12. The composition of claim 1, wherein the oral care composition is free of zinc.

13. The oral care composition of claim 1, wherein the oral care composition further comprises an amino acid, and the amino acid comprises glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine, or combinations thereof.

14. The composition of claim 1, wherein:
the dicarboxylic acid or salt thereof comprises from about 0.1% to about 15%, by weight of the oral care composition, of oxalic acid or a salt thereof,
the fluoride ion source comprises sodium monofluorophosphate, stannous fluoride, or a combination thereof, and
the oral care composition, after 12 months of aging at about 20° C., has a soluble fluoride level that is more than 60% of the theoretical amount of fluoride.

15. The composition of claim 14, wherein the composition has a pH in a range of from about 4.5 to about 5.5.

16. The composition of claim 14, wherein the composition is essentially free of sodium fluoride.

17. The composition of claim 14, wherein the fluoride ion source comprises sodium monofluorophosphate, and the oral care composition, after 12 months of aging at about 20° C., has a soluble fluoride level that is more than 70% of the theoretical amount of fluoride.

18. An oral care composition comprising:
(a) from about 0.1% to about 15%, by weight of the composition, of dicarboxylic acid or a salt thereof;
(b) from about 0.01% to about 10%, by weight of the composition, of a tin ion source;
(c) soluble fluoride; and
(d) silica abrasive,
wherein the composition has a pH in a range of from about 4 to about 5.5.

19. The composition of claim 18, wherein the oral care composition, after 12 months of aging at about 20° C., has a level of the soluble fluoride that is more than 50% of a theoretical amount of fluoride.

20. The oral care composition of claim 18, wherein the tin ion source comprises stannous fluoride, stannous chloride, or combinations thereof.

21. An oral care composition comprising:
(a) from about 0.1% to about 15%, by weight of the composition, of malonic acid or a salt thereof;
(b) sodium fluoride; and
(c) silica abrasive,
wherein the composition has a pH in a range of from about 4 to about 5.5.

22. The composition of claim 21, wherein the oral care composition, after 12 months of aging at about 20° C., has a soluble fluoride level that is more than 60% of a theoretical amount of fluoride.

23. The composition of claim 21, wherein the composition is essentially free of or free of an oxalic acid or a salt thereof.
